(19) United States
(12) Patent Application Publication
(10) Pub. No.: US 2019/0350666 A1
(43) Pub. Date: Nov. 21, 2019

(54) MEDICAL DEVICE FOR PERFORMING A BIOPSY AND METHOD FOR PRODUCING THE MEDICAL DEVICE

(71) Applicant: FRAUNHOFER-GESELLSCHAFT ZUR FOERDERUNG DER ANGEWANDTEN FORSCHUNG E.V., Muenchen (DE)

(72) Inventors: Ronny GRUNERT, Radebeul (DE); Marcel MUELLER, Dresden (DE); Sandra HUNGER, Dresden (DE)

(73) Assignee: FRAUNHOFER-GESELLSCHAFT ZUR FOERDERUNG DER ANGEWANDTEN FORSCHUNG E.V., Muenchen (DE)

(21) Appl. No.: 16/314,648

(22) PCT Filed: Jun. 23, 2017
(86) PCT No.: PCT/EP2017/065577

§ 371 Date: Dec. 31, 2018

(30) Foreign Application Priority Data
Jun. 30, 2016 (DE) 10 2016 211 969.9

(51) Int. Cl. A61B 90/14; A61B 90/11; A61B 10/02; A61N 1/05; A61N 5/06; A61B 8/12; B33Y 80/00; B33Y 50/02; B29C 64/393

(52) U.S. Cl. CPC A61B 90/14; A61B 90/11; A61B 10/0233; A61N 1/0534; A61N 5/0601; B33Y 10/00; A61B 8/12; B33Y 80/00; B33Y 50/02; B29C 64/393; A61N 2005/063; A61N 5/062

(57) ABSTRACT

The present invention relates to a medical device for performing a biopsy in a region (2) of a body to be examined (8) and a method for producing the medical device. The medical device has at least three connection elements (1) for attaching the device to the region (2) of the body (8) or for attaching the device to anchoring elements that are arranged on the region (2) of the body (8), a sleeve (3) as a guide for a medical instrument (6), and a connecting element (4), by means of which the at least three anchoring elements (1) are connected to the sleeve (3). At least the connecting element (4) is formed from a lightweight construction material.

The present invention relates to a medical device for performing a biopsy and a method for producing the medical device.

Brain biopsies are conducted in order to take samples of etiologically unclear, damaged tissue (a lesion) when other diagnostic measures have been exhausted, increasingly severe symptoms occur, or the lesion shows progressive growth. Removed tissue is histopathologically examined and is used after completion of the biopsy for further treatment and assessing the prognosis.

Stereotactic biopsies have been used since the middle of the last century in a virtually unchanged form. Possible complications that may occur during or following a brain biopsy included hemorrhaging near the sampling site, wound healing disorders, and wound infections. Moreover, swelling or edema may occur, which in the vicinity of the eloquent centers of the brain (e.g. the speech center or the motor center) can lead to minor to severe neurological impairment. It is therefore important to use a biopsy system that has a high degree of accuracy and simultaneously causes the least possible trauma to the intact surrounding tissue. This applies equally to biopsies in other parts of the body. For example, the document U.S. Pat. No. 5,387,220 A describes a stereotactic frame that uses natural reference points during an operation and does not have to be attached to the head by means of pins.

An established method of taking tissue samples in neurosurgery is currently a stereotactic brain biopsy using a fixing frame. In biopsies using fixing frames, planning data are prepared prior to surgery and further processed. The stereotactic frame is equipped with instruments the position of which can be modified. It is thus possible to adjust the setting for a position of a biopsy needle.

In addition, a biopsy can be accompanied by real time magnetic resonance imaging (MRI) images. In this case, the course of the biopsy needle can be followed by continuous image monitoring, and interventions can be carried out as needed. The method provides a high degree of accuracy, but is costly to carry out.

Biopsies in veterinary medicine are ordinarily carried out on a freehand basis, i.e. without using a stereotactic system. However, holding devices can be used in this case as well.

Nevertheless, the drawback of the above-described frame-based methods is that the size of the frame has an effect on manageability during the operation. In addition, the frame can cause the patient pain. The fixation can result in postoperative infections, and the duration of operations using biopsy frames is generally almost twice as long as in biopsies conducted without using a frame.

The object of the present invention is therefore to provide an economical and simple biopsy system that is not susceptible to error.

This object is achieved according to the invention by means of a medical device according to claim 1 and a method for producing the medical device according to claim 9. Advantageous embodiments and improvements are described in the dependent claims.

A medical device for performing a biopsy in a region of a body to be examined has at least three connection elements by means of which the device can be attached to the region of the body or to anchoring elements that are arranged on the region of the body. In addition, a sleeve that serves as a guide for a medical instrument and a connecting element are provided, by means of which the at least three anchoring elements are connected to the sleeve. At least the connecting element is formed from a lightweight construction material.

By means of the connection elements, the medical device can be reliably fixed in place on the region of the body to be examined. During surgical intervention, the region to be examined, for example the head of a person or animal, has unrestricted mobility. At the same time, extremely high positional accuracy can be achieved. Because the lightweight construction concept is implemented with the lightweight construction material, the medical device is extremely light, which increases wearing comfort. In addition, one can also work with smaller dimensions than would be possible with conventional systems. Preferably, the at least three connection elements and the sleeve are formed from the lightweight construction material.

It can be provided that the sleeve is designed such that an entry point or target point of the medical instrument in the region of the body coincides with the center of gravity of the medical device. This results in a particularly stable arrangement that minimizes any wearing discomfort for the patient.

The at least three connection elements can be designed for attachment to bone anchors, which serve as anchoring elements. By means of bone anchors, a fixed point of attachment on a bone can be selected, so that positioning of the medical device is simplified.

As a medical instrument, the medical device can comprise a drill for making a suitable insertion opening, a biopsy needle for performing the biopsy, at least one electrode for deep brain stimulation, at least one endoscope including a light guide cable for photodynamic therapy, and/or an ultrasonic probe that is typically configured to emit ultrasound waves. Alternatively or additionally, the connecting element can be configured in a web-like manner, i.e. in particular can have a length that is greater by at least 10 percent than its width and/or thickness. Typically, both the length and the width, as well as the thickness of the connecting element, cannot be changed, but are predetermined or fixed.

The lightweight construction material is typically selected from plastic, preferably a polycarbonate (PC), a polyamide (PA) and/or acrylonitrile-butadiene-styrene (ABS), aluminum, titanium, an alloy of the aforementioned elements, or a fiber composite material, which preferably comprises one or more of these materials. These materials are easy to process, and at the same time offer the possibility of producing a mechanically stable device for performing the biopsy.

The connecting element can be designed to be rigid in order to increase the mechanical stability. In particular, the dimensions of the connecting element should be unchangeable.

Alternatively or additionally, the connecting element can be integrally formed, i.e., can be composed of a single piece, which also increases mechanical stability. Particularly preferably, the connecting element, the sleeve, and/or at least one of the anchoring elements, typically all of the anchoring elements, are integrally formed and preferably connected to one another in a positively interlocking manner.

A method for producing the above-described medical device includes a step in which an image of the region of the body is prepared by means of magnetic resonance imaging, computed tomography, and/or ultrasound images. After this, the medical device is generated in that coordinates of anchoring elements arranged on the region of the body or coordinates of points of attachment in the region of the body for connection elements are determined in the image, the corresponding connection elements are constructed, a target point or entry point is defined as the impact point of the medical instrument, a position of the sleeve is specified, and at least one connecting element is provided. Finally, the above-defined device is produced by a rapid prototyping method, a computer-aided design (CAD) method, and/or a computer-aided manufacturing (CAM) method.

Because a rapid prototyping method, a CAD method, or an CAM method is used, the described medical device can be produced rapidly and economically, and because of the adaptation to the respective patient, no sterilization is required after the operation. Typically, the medical device is designed for single use, so there are no restrictions on use in particular diseases such as Creutzfeldt-Jacob disease or slow viral infections/prions.

In each case, an image is preferably prepared by means of magnetic resonance imaging and computed tomography of the region of the body, wherein the two images are then merged together. The merged image is then used to generate the device. By means of magnetic resonance imaging, for example, target points in the brain can be specified, and this method also allows highly favorable soft tissue recognition and distinction of intact tissue. The computed tomography in turn makes it possible to detect bony landmarks and bone anchors.

The merging can be carried out using anatomical and/or predeterminable, typically artificial or man-made reference points. The reference points, for example the bone anchors, are assigned to the region of the body to be more closely examined.

Prior to production by the rapid prototyping method or the computer-aided design/computer-aided manufacturing method, a fillet can be provided in generating the medical device by means of the image in order to simplify the handling of the medical device. Alternatively or additionally, at least one threaded hole is provided for attaching further holding devices or units.

For the rapid prototyping method, depending on the material used, polyamide casting, binder jetting, stereolithography, fused deposition modeling, multi jet modeling, selective laser melting, or selective laser sintering should be used.

A computer program product can comprise a computer program that has software means for performing the above-described method, if the computer program in carried out or executed on an automation system.

An example of the invention is shown in the drawing and will be explained in the following with reference to FIGS. 1 to 4.

The figures show the following:

FIG. 1 a perspective view of a head with the medical device for a biopsy;

FIG. 2 a side view of a marker;

FIG. 3 a perspective view of a bone screw, and

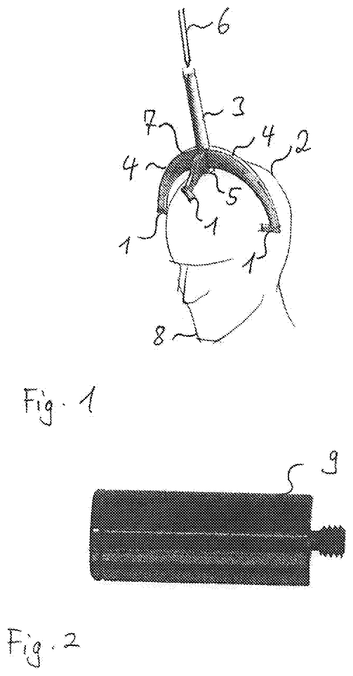

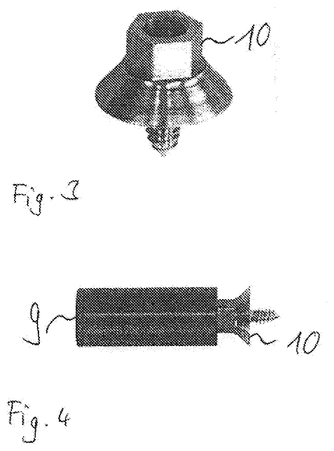

FIG. 4 the marker connected to the bone screw in a view corresponding to FIG. 2.

FIG. 1 is a perspective view of a head 2 as the region of a body 8 to be examined. In further examples, however, another body part can also be provided for a biopsy by means of the device described in further detail. For fixation of a target device to be produced, three bone anchors have already been attached to the head 2 or skull as part of the biopsy procedure to be performed or prior to the biopsy procedure to be performed. The three bone anchors are attached to the outer table of the bony skull (the occipital, temporal, and zygomatic bones bilaterally) and serve both as artificial landmarks and contact points. The number of bone anchors to be used, also referred to as fixation anchors, is based on a compromise between fastening stability and the weight of the medical device. However, the number should be at least three in order to ensure sufficient stability. Typically, exactly three bone anchors are placed. If there are too few points of attachment, this results in unstable attachment, while too many points of attachment increase the weight and complexity of the medical device, which adversely affects its accuracy. It can also be provided that anchoring elements or connection elements 1 are noninvasively placed only in the region in question.

After thorough planning by a surgeon, the bone anchors are screwed into the skull at the positions shown in FIG. 1. The placement depends on the location of the tumor in the brain. However, an effort is made to determine standardized points for typical tumor locations. Because of subsequent preoperative data collection by means of computed tomography (CT) and/or magnetic resonance imaging (MRI), the reference points for construction of the target device at the planned target and entry point 5 are known. Alternatively, only one CT image or only one MRI image is produced.

The data contained in the CT image and the MRI image can be merged with each other by means of anatomical and/or artificial landmarks. In the individual or merged image, target and entry coordinates are established that determine the entry point 5 of the medical instrument 6, for example a medical drill or a biopsy needle. In the example shown, the entry point 5 coincides with the center of gravity of the completed medical device. Finally, the biopsy is localized, sparing eloquent regions and high-risk structures.

Based on the given parameters, such as the coordinates of the connection elements 1 or anchoring elements and the entry point 5, the medical device is finally constructed. This can be done automatically. For this purpose, for example using a suitable computer program, a biopsy needle is constructed in the example shown from the coordinates of the target point and entry point 5. After this, the connection elements 1 are added. The target device, in the example shown a sleeve 3 for a drill or the biopsy needle, is arranged in the example shown in the center between the connection elements 1 and connected to the connection elements 1 in each case by means of a web-like connecting element 4. The sleeve 3 is constructed as a needle guide with a defined length, and the exactly three connection elements 1 are constructively connected by the connecting elements 4 to the sleeve 3 as a needle guide element. Finally, fillets 7 and threaded holes are added, for example in order to adapt the shape of the connecting elements 4 to the shape of the head and thus obtain the most compact medical device possible. The connection elements 1, the connecting elements 4, and the sleeve 3 are integrally formed in the example shown and in each case are rigidly connected to one another. In particular, none of the above-mentioned elements can undergo a tilting or rotary movement without a corresponding simultaneous movement of another of the elements. The above-mentioned elements are also rigidly connected to one another. In each case in the example shown, one of the connecting elements 4 connects the sleeve to one individual connection element 1.

The medical device virtually constructed in this manner is now produced by means of a rapid prototyping method. For this purpose, the medical device virtually constructed by means of a computer program can be transferred as a data set to a calculating unit such as a computer, wherein the computer program software comprises means for carrying out the above-described method if the computer program is executed on the computer as an automation system. The computer then controls a device for performing the rapid prototyping method, the CAD method, and/or the CAM method. In particular, for the rapid prototyping method, one can use polyamide casting, binder jetting, stereolithography, fused deposition modeling, multi jet modeling, selective laser melting, or selective laser sintering, with processes being carried out depending on the method used with a plastic, aluminum, magnesium, titanium, an alloy comprising aluminum, magnesium, or titanium, or a fiber composite material. Here, at least the connecting elements 4 are composed of a lightweight construction material, while in further examples the sleeve 3 and/or the connection elements 4 can also be composed of high-weight materials. Preferably, however, the sleeve 3 and the connection elements 4 are formed from the same material as the connecting elements 4. Using the lightweight construction concept implemented in this manner, for example, one obtains a medical device that is extremely light, with a weight of between 200 g and 250 g, typically 300 g, and is significantly lighter than a comparable stereotactic frame, which ordinarily weighs 3 kg. In addition, this device can be prepared for use by steam sterilization.

It should only be necessary to wear the device during the operation, wherein the duration of surgery is also shortened because of the simplified handling. All of the settings are already implemented by the rapid prototype production, thus obviating the need for time-consuming adjustments.

Alternatively or additionally to guidance of the biopsy needle, the device can also be used for the placement of at least one, and preferably at least two electrodes for deep brain stimulation, for photodynamic therapy, in which an endoscope and an optical waveguide are inserted, and/or for treatment by means of pulsed ultrasound.

In an example, only a single MRI image is made, for which markers are used that are fastened to the bone anchors, i.e. the subsequent connection sites of the medical device to be produced to the cranial bones, for the period of magnetic resonance imaging. These markers, which are typically composed of a material suitable for magnetic resonance imaging, are attached to the available bone, for example by means of screws. For this purpose, the markers have a cylindrical body that is filled with the medium. A screw thread is attached to one of the two surfaces adjacent to an outer surface of the cylindrical body. FIG. 2 shows a side view of a corresponding marker 9.

FIG. 3 shows a perspective view of a bone screw 10, also referred to as a bone anchor, which on its underside also has a cutting thread by means of which it can be attached to the patient. Opposite to the cutting thread is an internal screw thread designed such that the screw thread of the marker 9 can be secured therein. Because of the known geometry of the marker 9 and the coordinate points in space that can be determined based on this geometry, the medical device can be rapidly constructed and produced by a rapid prototyping method and/or a computer-aided design/computer-aided manufacturing method. In this manner, only one imaging method, namely computed tomography or magnetic resonance imaging, is necessary in order to plan the biopsy, so that the inaccuracies in locating a target point that can occur in merging processes can be prevented.

FIG. 4 shows how the marker 9 is connected to the bone screw 10 in a view corresponding to FIG. 2.

Features of the individual embodiments disclosed only in the examples can be combined with one another and individually claimed.

1. A medical device for performing a biopsy in a region (2) of a body to be examined (8) with
at least three connection elements (1) for attaching the device to the region (2) of the body (8) or for attaching the device to anchoring elements that are arranged on the region (2) of the body (8),
a sleeve (3) as a guide for a medical instrument (6), and
a connecting element (4), by means of which the at least three anchoring elements (1) are connected to the sleeve (3), wherein
at least the connecting element (4) is formed from a lightweight construction material.

2. The medical device according to claim 1, characterized in that the sleeve (3) is designed such that an entry point (5) of the medical instrument in the region (2) of the body (8) coincides with the center of gravity of the medical device.

3. The medical device according to claim 1, characterized in that the at least three connection elements (1) are designed for attachment to bone anchors as anchoring elements.

4. The medical device according to claim 1, characterized by including a drill, a biopsy needle, at least one electrode for deep brain stimulation, at least one endoscope including a light guide cable for photodynamic therapy, and/or an ultrasonic probe as the medical instrument (6).

5. The medical device according to claim 1, characterized in that the lightweight construction material is selected from plastic, preferably a polycarbonate, a polyamide and/or acrylonitrile-butadiene-styrene, aluminum, titanium, an alloy of these elements or a fiber composite material.

6. The medical device according to claim 1, characterized in that the connecting element (4) is rigid.

7. The medical device according to claim 1, characterized in that the connecting element (4) is integrally formed.

8. The medical device according to claim 1, characterized in that the connecting element (4) is connected in a positively locking manner with the sleeve (3) and/or the anchoring elements (1).

9. A method for producing a medical device according to claim 1, wherein
an image of the region (2) of the body (8) is made by means of magnetic resonance imaging, computed tomography and/or ultrasound images,
the device is generated in that coordinates of anchoring elements arranged on the region (2) of the body (8), or coordinates of points of attachment in the region (2) of the body (8) for connection elements (1), are determined in the image and the connection elements (1) are constructed,
an entry point and target point (5) is defined as an impact point of the medical instrument (6),
a position of the sleeve (3) is specified and at least one connecting element (4) is provided, and
finally, the above-defined device is produced by a rapid prototyping method and/or a computer-aided design/computer-aided manufacturing method.

10. The method according to claim 9, characterized in that in each case, an image is made by magnetic resonance imaging and computed tomography of the region (2) of the body (8) and the two images are then merged.

11. The method according to claim 10, characterized in that the merging is carried out based on anatomical and/or predeterminable reference points arranged on the region (2) of the body (8).

12. The method according to claim 9, characterized in that before production by the rapid prototyping method and/or the computer-aided design/computer-aided manufacturing method, at least one fillet (7) and/or at least one threaded hole is/are provided.

13. The method according to claim 9, characterized in that polyamide casting, binder jetting, stereolithography, fused deposition modeling, multi jet modeling, selective laser melting, or selective laser sintering is used as the rapid prototyping method.

14. A computer program product with a computer program comprising software means for performing the method according to claim 9 when the computer program is executed on an automation system.
